(19) United States
(12) Patent Application Publication
(10) Pub. No.: US 2022/0110668 A1
(43) Pub. Date: Apr. 14, 2022

(54) INTRODUCER FOR COUPLING WITH ABLATION PROBES

(71) Applicants: Focused Cryo, Inc., Kennesaw, GA (US); Emory University, Atlanta, GA (US)

(72) Inventors: Yogi Patel, Kennesaw, GA (US); John David Prologo, Alpharetta, GA (US)

(21) Appl. No.: 17/501,366

(22) Filed: Oct. 14, 2021

Related U.S. Application Data
(60) Provisional application No. 63/091,481, filed on Oct. 14, 2020.

(51) Int. Cl. A61B 18/02

(52) U.S. Cl. CPC A61B 18/02; A61B 2018/00577; A61B 2018/0293

(57) ABSTRACT

Introducers for detachably coupling with an ablation probe are described herein. The ablation probe can be a cryoablation probe or other type of ablation probe such as microwave or RF ablation probe. An example introducer includes a hollow member; and a locking mechanism configured to secure the introducer and the ablation probe. An example system includes an ablation probe; and an introducer that is configured to detachably couple with the ablation probe. The introducer includes a hollow member, one or more energy elements arranged along an axial direction of the hollow member, and one or more sensor elements arranged along the axial direction of the hollow member.

### CROSS-REFERENCE TO RELATED APPLICATIONS

This application claims the benefit of U.S. provisional patent application No. 63/091,481, filed on Oct. 14, 2020, and titled “INTRODUCER FOR COUPLING WITH ABLATION PROBES,” the disclosure of which is expressly incorporated herein by reference in its entirety.

### BACKGROUND

Cryoneurolysis procedures attempt to ablate specific anatomical tissues to treat a variety of chronic disorders. The target anatomical tissues can be of various geometries, be at various locations relative to other organs, and be under significantly different thermal stresses depending upon the patient's body composition. To achieve targeted, complete, and effective cryoneurolysis (or cryoablation) the probe geometries must be designed to enable appropriate contact with the tissues. In many cases, these geometries can be complex with structures that are not easy to pass through the skin, organs, and nearby tissues to reach the target.

### SUMMARY

An introducer that is configured to detachably couple with an ablation probe is described herein. The introducer includes a hollow member; and a locking mechanism configured to secure the introducer and the ablation probe.

Additionally, the introducer includes one or more energy elements arranged along an axial direction of the hollow member, and one or more sensor elements arranged along the axial direction of the hollow member. Optionally, the introducer further includes a circuit board, and the one or more energy elements and/or the one or more sensor elements are disposed on the circuit board.

Optionally, in some implementations, the introducer further includes an inner removable core.

Optionally, in some implementations, the introducer further includes a coupling sensor configured to detect coupling of the introducer to the ablation probe.

Optionally, in some implementations, the introducer further includes a stabilization mechanism configured to maintain positioning of the introducer.

Optionally, in some implementations, the introducer further includes a fiducial marker.

Optionally, in some implementations, the introducer further includes a visual indicator.

Optionally, in some implementations, the introducer further includes an inertial sensor.

Optionally, in some implementations, the introducer further includes an electromagnetic sensor.

Optionally, in some implementations, the introducer further includes a controller. The controller is configured to spatially and temporally control an ablation zone.

An introducer system is also described herein. The system includes an ablation probe; and an introducer that is configured to detachably couple with the ablation probe. The introducer includes a hollow member, one or more energy elements arranged along an axial direction of the hollow member, and one or more sensor elements arranged along the axial direction of the hollow member. Optionally, the introducer further includes a circuit board, and the one or more energy elements and/or the one or more sensor elements are disposed on the circuit board.

Optionally, in some implementations, the introducer further includes an inner removable core.

Optionally, in some implementations, the introducer further includes a locking mechanism configured to secure the introducer and the ablation probe.

Optionally, in some implementations, the introducer further includes a coupling sensor configured to detect coupling of the introducer to the ablation probe.

Optionally, in some implementations, the introducer further includes a stabilization mechanism configured to maintain positioning of the introducer.

Optionally, in some implementations, the introducer further includes a fiducial marker.

Optionally, in some implementations, the introducer further includes a visual indicator.

Optionally, in some implementations, the introducer further includes an inertial sensor.

Optionally, in some implementations, the introducer further includes an electromagnetic sensor.

Optionally, in some implementations, the system further includes a controller operably connected to the introducer. The controller is configured to spatially and temporally control an ablation zone.

Optionally, the ablation probe is a cryoablation probe.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A illustrates an example intelligent introducer system according to an implementation described herein. FIG. 1B illustrates a radial cross section of the introducer along line A-A′ in FIG. 1A.

FIG. 2A illustrates another view of the intelligent introducer system of FIG. 1A.

FIG. 2B illustrates a radial cross section of the introducer and ablation probe along line A-A′ in FIG. 2A.

FIG. 3 illustrates a radial cross section of an example introducer system according to an implementation described herein.

FIG. 4 is an example computing device.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “about” or “approximately” when referring to a measurable value such as an amount, a percentage, and the like, is meant to encompass variations of ±20%, ±10%, ±5%, or ±1% from the measurable value.

An intelligent introducer system that couples with any third party cryoablation or ablation probe/catheter is described herein. Example cryoablation systems are described in WO2020/163854, the disclosure of which is expressly incorporated herein by reference in its entirety. The resulting coupled introducer and ablation probe/catheter enables physicians to target specific tissues, control the temperature, and obtain direct feedback on the state and progress of the ablation procedure. The introducer comprises electronics, material coatings, and components that measures, controls, and provides direct insight into the status of the procedure. In some implementations, the system comprises the introducer, a marker for image recognition and processing, and a control module that communicates to the physician through a computing device such as a tablet or other computer unit.

In one example implementation, the intelligent introducer system includes an ablation probe, and an introducer that is configured to detachably couple with the ablation probe. This disclosure contemplates that the ablation probe may be any third party cryoablation or other ablation (microwave, RF, etc.) probe/catheter. Optionally, in some implementations, the ablation probe is a cryoablation probe.

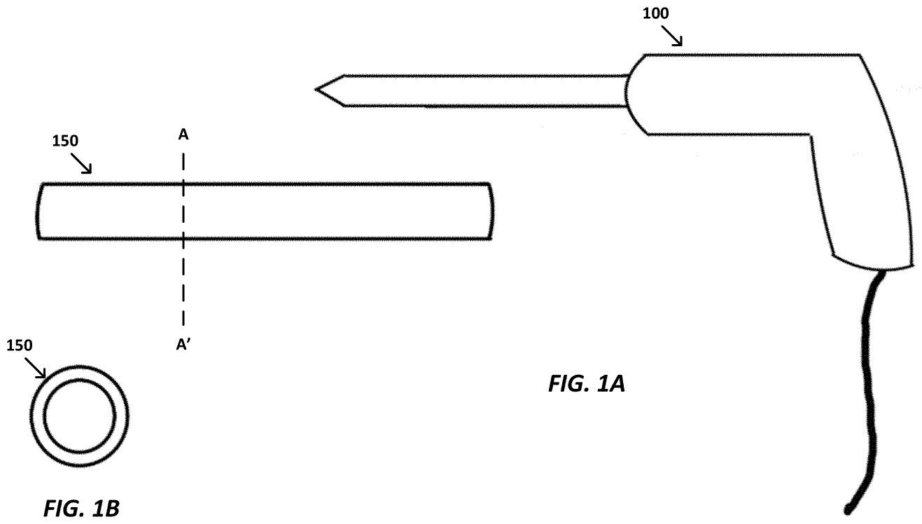

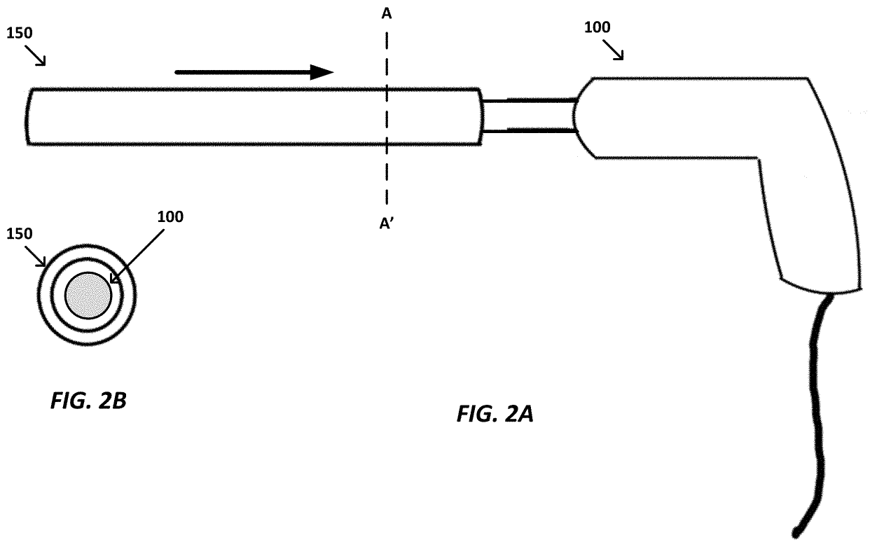

Referring now to FIGS. 1A-2B, an example intelligent introducer system according to implementations described herein are shown. The system includes an ablation probe 100 and an introducer 150. As described herein, the ablation probe 100 can optionally be a cryoablation probe. It should be understood that the ablation probe 100 can be another type of probe including, but not limited to, microwave or radiofrequency (RF) ablation probes. Ablation probes are well known in the art and therefore not described in further detail herein. As noted above, this disclosure contemplates that the ablation probe can be an ablation probe/catheter known in the art. In the implementations of FIGS. 1A-2B, the introducer 150 and probe 100 are configured for detachable coupling. For example, in FIG. 1A, the introducer 150 and probe 100 are detached (i.e., not coupled). And in FIG. 2A, the introducer 150 and probe 100 are partially coupled. The introducer 150 and probe 100 are coupled by positioning the introducer 150 around the probe 100, for example, as shown by the arrow in FIG. 2A. The introducer 150 includes a hollow tube (see radial cross section view of FIG. 1B). Thus, when coupled to the probe 100, the introducer 150 surrounds the probe 100 (see radial cross section view of FIG. 2B). The introducer 150 includes a locking mechanism configured to secure the introducer 150 and the ablation probe 100. The locking mechanism can be a pin, latch, screw, magnet, or other mechanism configured to secure the introducer 150 and the ablation probe 100. In some implementation, the locking mechanism extends/retracts from the introducer 150 to engage the ablation probe 100. For example, this disclosure contemplates that the locking mechanism can be engaged, manually or automatically, to keep the introducer 150 in a fixed position relative to the ablation probe 100. This disclosure also contemplates that the locking mechanism can be disengaged, manually or automatically, to decouple the introducer 150 from the ablation probe 100.

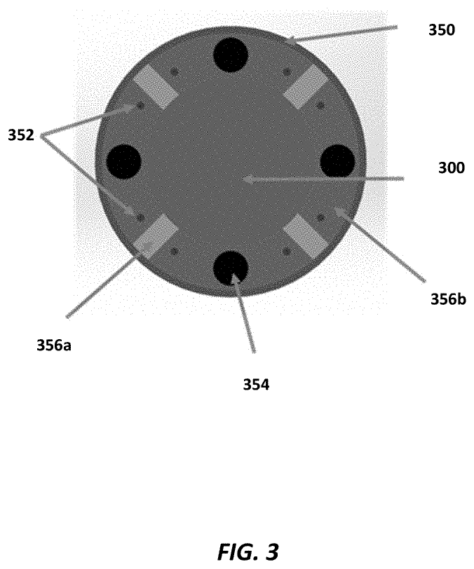

Additionally, the introducer 150 includes one or more energy elements (see FIG. 3) arranged along an axial direction of the hollow member, and one or more sensor elements (see FIG. 3) arranged along the axial direction of the hollow member. Optionally, the introducer 150 further includes a circuit board, and the one or more energy elements and/or the one or more sensor elements are disposed on the circuit board. Energy elements and sensors are described in WO2020/163854, the disclosure of which is expressly incorporated herein by reference in its entirety.

Optionally, in some implementations, the introducer 150 further includes an inner removable core.

Optionally, in some implementations, the introducer 150 further includes a coupling sensor configured to detect coupling of the introducer 150 to the ablation probe 100. This disclosure contemplates using any sensor that can detect coupling of the introducer 150 and probe 100, e.g., magnetic sensors, strain sensors, etc.

Optionally, in some implementations, the introducer 150 further includes a stabilization mechanism configured to maintain positioning of the introducer 150. For example, the stabilization mechanism can be a component or portion of the introducer 150 that extends radially toward the ablation probe 100 when coupled. This disclosure contemplates that the stabilization mechanism can be made of any suitable material including, but not limited to, rigid and elastic materials.

Optionally, in some implementations, the introducer 150 further includes one or more surgical navigation sensors. Such sensors can include, but are not limited to, an inertial sensor (e.g., accelerometer, gyroscope, magnetometer, or combinations thereof) or electromagnetic sensor. It should be understood that inertial and electromagnetic sensors are provided only as examples. This disclosure contemplates using other types of sensors for surgical navigation.

Optionally, in some implementations, the introducer 150 further includes a fiducial marker (e.g., a reflective material, barcode, or other visual marking) and/or a visual indicator (e.g., a light emitting diode or light source). Such markers or indicators can be used for guidance during a surgical procedure.

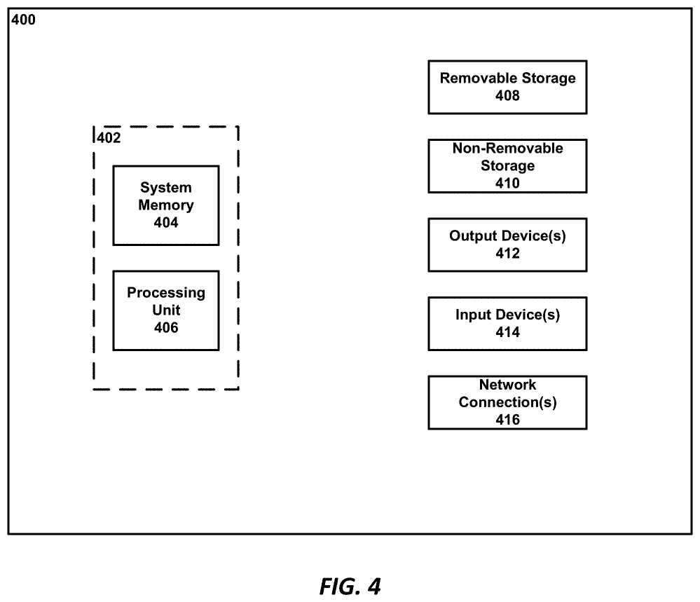

Optionally, in some implementations, the introducer 150 further includes a controller. The introducer 150 can be coupled to the controller (e.g., computing device of FIG. 4) through one or more communication links. This disclosure contemplates the communication links are any suitable communication link. For example, a communication link may be implemented by any medium that facilitates data exchange between the introducer 150 and controller including, but not limited to, wired, wireless and optical links. The controller is configured to spatially and temporally control an ablation zone. For example, the introducer 150 can communicate with the controller. As described below, the controller is configured to receive measurements from one or more of the sensor elements of the introducer 150. Alternatively or additionally, the controller is configured to control (e.g., turn ON/OFF) one or more of the energy elements of the introducer 150. Spatial and temporal control of the ablation zone is described in are described in WO2020/163854, the disclosure of which is expressly incorporated herein by reference in its entirety.

Referring to FIG. 3, the introducer includes a hollow member 350, one or more energy elements 352, and one or more sensor elements 354 (e.g., sensors). Additionally, the one or more energy elements 352 and one or more sensor elements 354 are optionally separated by insulating or conducting materials 356a, 356b. As shown in FIG. 3, the ablation probe 300 is inserted into the hollow member 350 of the introducer. The energy element(s) 352 and the sensor element(s) 354 are arranged along an axial direction of the hollow member 350 (not shown in FIG. 3). An energy element 352 can be configured to convert electrical energy to heat or cold. A sensor element 354 can be configured to measure temperature in proximity to the introducer. It should be understood that temperature sensing is provided only as an example. This disclosure contemplates that the sensor element 354 can be configured to measure other parameters including, but not limited to, electrical current, chemical element, or tissue sample or tissue characteristics. In some implementations, the introducer includes both energy elements and sensor elements. In other implementations, the introducer may include only energy element(s). In yet other implementations, the introducer may include only sensor element(s). It should be understood that the number, spacing, and arrangement of the energy elements 352 and sensor elements 354 in FIG. 3 are provided only as an example. This disclosure contemplates providing an introducer having different numbers and/arrangements of energy elements 352 and sensors 354.

In some implementations, the system further includes a controller (e.g., computing device of FIG. 4) operably connected to the introducer. The introducer can be coupled to the controller through one or more communication links. This disclosure contemplates the communication links are any suitable communication link. For example, a communication link may be implemented by any medium that facilitates data exchange between the introducer and controller including, but not limited to, wired, wireless and optical links. The controller is configured to receive measurements from one or more of the sensor elements 354. Alternatively or additionally, the controller is configured to control (e.g., turn ON/OFF) one or more of the energy elements 352. As such, the controller can be configured to spatially and temporally control a ablation zone, for example, by individually addressing and controlling one or more of the energy elements 352. In some implementations, the controller can be configured to use real-time temperature feedback as measured by the one or more sensing elements 354 to control the energy elements 352.

This disclosure contemplates that the intelligent introducer and/or intelligent introducer system can include one or more of the following features:

The system can be configured for measurement of temperature at least one physical points in the patient.

The system can be configured for steering biasing of the ice with at least 90 degrees (left or right of the probe).

The introducer can include a hollow tube with inner removable core with locking mechanism for attaching to any 3rd party cryoablation or other ablation (microwave, RF, etc.) probe/catheter.

The introducer can have an electromechanical sensing interface to validate coupling of ablation probe with introducer.

The introducer can have a mechanical locking mechanism for coupling ablation probe with introducer.

The introducer can have a stabilization mechanism for maintaining position after placement.

The introducer can have one or more sensing elements for detection of orientation, position, and motion.

The introducer can be of single element fabrication where multiple components are individually placed for desired function or can have a single packaged component with all elements placed in a circuit board or thin film flexible circuit.

The introducer can be used for cryoablation, microwave ablation, radiofrequency ablation, or any other energy based ablation mechanism.

The introducer geometry can be a cylinder or other polygonal (e.g., hexagon, pentagon, etc.) structure.

The introducer can be hollow on one end or have a tip.

The introducer can have an inner removable component with a tip, which after removal provides a hollow end for insertion of devices.

The introducer can be used to place biopsy needle for sampling.

The introducer can be used to coagulate or cauterize tissue/tract.

The introducer can be made of metals, polymers, hydrogels, ceramic, or other materials.

The system can include a controller, and the introducer can be coupled to the controller, for example by a wired or wireless link, and communicate with the controller. For example, the introducer can send data and receive data from controller. The controller communicates data through wired or wireless link with a tablet or other computer system. Alternatively or additionally, the controller interfaces with hospital network, CT scanner, or other imaging equipment for real-time acquisition of images. The tablet provides real-time visualization of procedure status and measurements. The tablet provides real-time control of introducer functionality.

The introducer can have a circuit board with energy producing elements.

The introducer can have energy producing elements can generate heat or generate cold.

The introducer can have a circuit board with temperature measurement elements.

The introducer can have a ring of electrode contacts for detection of ice generation during cryoablation procedures.

In the case of cryoablation, the introducer can detect generation of ice and measure the temperature of the ice generation.

In the case of cryoablation, the introducer can augment the ablation zone shape and size by energizing individual energy producing elements within the introducer.

In the case of heat based ablation (e.g., microwave, radiowave, etc.), the introducer can detect generation of the ablation zone and measure the ablation zone temperature.

In the case of heat based ablation, the introducer can augment the ablation zone shape and size by energizing individual energy producing elements within the introducer.

The introducer can have electromagnetic sensors for position and guidance tracking.

The introducer can have can be of varying diameters to support variable probe diameters.

The introducer can have visual markings to provide visual feedback to the user on the orientation of the introducer.

The introducer can have visual indicators (e.g., light emitting diodes) to provide visual feedback to the user on the status of communication with the software and controller.

It should be appreciated that the logical operations described herein with respect to the various figures may be implemented (1) as a sequence of computer implemented acts or program modules (i.e., software) running on a computing device (e.g., the computing device described in FIG. 4), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 4, an example computing device 400 upon which the methods described herein may be implemented is illustrated. It should be understood that the example computing device 400 is only one example of a suitable computing environment upon which the methods described herein may be implemented. Optionally, the computing device 400 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 400 typically includes at least one processing unit 406 and system memory 404. Depending on the exact configuration and type of computing device, system memory 404 may be volatile (such as random access memory (RAM)), non-volatile (such as read-only memory (ROM), flash memory, etc.), or some combination of the two. This most basic configuration is illustrated in FIG. 4 by dashed line 402. The processing unit 406 may be a standard programmable processor that performs arithmetic and logic operations necessary for operation of the computing device 400. The computing device 400 may also include a bus or other communication mechanism for communicating information among various components of the computing device 400.

Computing device 400 may have additional features/functionality. For example, computing device 400 may include additional storage such as removable storage 408 and non-removable storage 410 including, but not limited to, magnetic or optical disks or tapes. Computing device 400 may also contain network connection(s) 416 that allow the device to communicate with other devices. Computing device 400 may also have input device(s) 414 such as a keyboard, mouse, touch screen, etc. Output device(s) 412 such as a display, speakers, printer, etc. may also be included. The additional devices may be connected to the bus in order to facilitate communication of data among the components of the computing device 400. All these devices are well known in the art and need not be discussed at length here.

The processing unit 406 may be configured to execute program code encoded in tangible, computer-readable media. Tangible, computer-readable media refers to any media that is capable of providing data that causes the computing device 400 (i.e., a machine) to operate in a particular fashion. Various computer-readable media may be utilized to provide instructions to the processing unit 406 for execution. Example tangible, computer-readable media may include, but is not limited to, volatile media, non-volatile media, removable media and non-removable media implemented in any method or technology for storage of information such as computer readable instructions, data structures, program modules or other data. System memory 404, removable storage 408, and non-removable storage 410 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory 404. For example, the bus may carry data to the system memory 404, from which the processing unit 406 receives and executes instructions. The data received by the system memory 404 may optionally be stored on the removable storage 408 or the non-removable storage 410 before or after execution by the processing unit 406.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. An introducer that is configured to detachably couple with an ablation probe, the introducer comprising:
a hollow member; and
a locking mechanism configured to secure the introducer and the ablation probe.

2. The introducer of claim 1 further comprising:
one or more energy elements arranged along an axial direction of the hollow member, and
one or more sensor elements arranged along the axial direction of the hollow member.

3. The introducer of claim 2, further comprising a circuit board, wherein the one or more energy elements or the one or more sensor elements are disposed on the circuit board.

4. The introducer of claim 1, further comprising an inner removable core.

5. The introducer of claim 1, further comprising a coupling sensor configured to detect coupling of the introducer to the ablation probe.

6. The introducer of claim 1, further comprising a stabilization mechanism configured to maintain positioning of the introducer.

7. The introducer of claim 1, further comprising a fiducial marker.

8. The introducer of claim 1, further comprising a visual indicator.

9. The introducer of claim 1, further comprising an inertial sensor.

10. The introducer of claim 1, further comprising an electromagnetic sensor.

11. The introducer of claim 1, further comprising a controller comprising a processor and a memory, the memory having computer-executable instructions stored thereon that, when executed by the processor, cause the controller to spatially and temporally control an ablation zone.

12. A system comprising:
an ablation probe; and
an introducer that is configured to detachably couple with the ablation probe, wherein the introducer comprises:
a hollow member,
one or more energy elements arranged along an axial direction of the hollow member, and
one or more sensor elements arranged along the axial direction of the hollow member.

13. The system of claim 12, wherein the introducer further comprises an inner removable core.

14. The system of claim 12, wherein the introducer further comprises a locking mechanism configured to secure the introducer and the ablation probe.

15. The system of claim 12, wherein the introducer further comprises a coupling sensor configured to detect coupling of the introducer and the ablation probe.

16. The system of claim 12, wherein the introducer further comprises a stabilization mechanism configured to maintain positioning of the introducer.

17. The system of claim 12, wherein the introducer further comprises a fiducial marker or visual indicator.

18. The system of claim 12, wherein the introducer further comprises an inertial sensor or an electromagnetic sensor.

19. The system of claim 12, further comprising a controller operably connected to the introducer, the controller comprising a processor and a memory, the memory having computer-executable instructions stored thereon that, when executed by the processor, cause the controller to spatially and temporally control an ablation zone.

20. The system of claim 12, wherein the ablation probe is a cryoablation probe.
